(19) United States
(12) Patent Application Publication
(10) Pub. No.: US 2005/0222169 A1
(43) Pub. Date: Oct. 6, 2005

(54) Compositions and methods of treating infections

(76) Inventors: Nawaz Ahmad, Monmouth Junction, NJ (US); Kalpana J. Patel, West Windsor, NJ (US); Brinda Wiita, Princeton, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/034,289

(22) Filed: Jan. 12, 2005

Related U.S. Application Data
(60) Provisional application No. 60/537,154, filed on Jan. 16, 2004.

(51) Int. Cl. A61N 1/00; A61K 31/7048; A61K 31/496; A61K 31/4196; A61K 31/4178

(52) U.S. Cl. 514/254.07; 514/383; 514/397; 607/1; 514/28; 514/169; 424/667

(57) ABSTRACT

This invention relates to methods, compositions and treatment regimens for applying cooling active ingredients to the perineum of a woman to treat the symptoms of a vaginal or vulvar infection or vulvar pain in order to speed the woman's relief from pain and/or itch.

### BACKGROUND OF THE INVENTION

For many years, women have suffered from vaginal infections that cause itching, pain and general discomfort to sufferers. In particular, women have suffered from vaginal yeast infections caused by the organism Candida albicans. If not treated promptly and appropriately, such vaginal yeast infections cause considerable itching, pain and discomfort. Conventional treatments of vaginal yeast infections include local application of creams, suppositories, soft gelatin capsules, vaginal tablets and vaginal ointments. These treatments are generally available in treatment regimens ranging from one to seven days. More recently, oral administration of antifungal compositions, such as Diflucan® tablets, has been available to patients for systemic treatment.

In many cases, vaginal yeast infections are not restricted to the vaginal area only. In fact, the infections may extend and involve the external vulvar area as well, causing discomfort and itching externally as well as internally. Various combination regimens have become available to treat such infections both internally and externally including creams, suppositories or ovules intended for internal insertion and creams intended for external application to the vulva to relieve itching, pain and discomfort in the vulvar area.

While women suffering the pain and discomfort of vaginal yeast infections may find relief eventually during the course of treatment with known regimens, they may not perceive this relief for several days. Although the application of external vaginal creams may offer some relief during the course of treatment, this relief is not immediate or instantaneous. The infection continues to produce a lumpy, cheesy discharge that heightens the discomfort caused by the infection.

### SUMMARY OF THE INVENTION

In view of the foregoing discussion, there is currently a need for a treatment regimen for vaginal infections that addresses both the need to eliminate the disease-causing organism and to ameliorate the suffering of the patient. We have discovered that cooling the area in or around which the infection is causing discomfort surprisingly helps to reduce the suffering of a patient during the course of a vaginal infection.

We theorize that the application of cold to or around the area which the infection affects reduces the transmittal of painful sensation along the nerve endings of these painful or injured tissues. This causes an actual reduction in pain. Furthermore, cooling the tissues reduces swelling in the injured muscles and tissues. In addition, application of cold to or around the area of infection, reduces blood flow and can hence reduce the extracellular discharge which can lead to reduction of inflammation and alleviation of symptoms.

The cooling process may be accomplished according to one or more of several methods. The physical application of a cold object or device to or around the area may accomplish the desired goals. For example, a cold pack containing ice or cloths that have been refrigerated may be applied during treatment to cool the area. Likewise, a cold pack wrapped in fabric and containing chemicals that endothermically react to become cold may also be applied.

However, such methods may be unwieldy or uncomfortable and may require the individual to remain stationary in private for some period of time. Thus, anther method by which the area may be cooled includes utilizing the heat of the infected tissues to evaporate components in a cooling device. Examples of this method include the application of water or alcohol or combinations thereof to the affected area.

Another method of cooling the vaginal and vulvar areas may include utilizing the cooling effect of evaporation of moisture upon applying moisture or a wet substrate to the areas. The moisture will then evaporate, causing the patient to experience a cooling sensation. Various methods of application of moisture can be used, such as a wet substrate, a device containing a moist substrate or sponge or the like.

Cooling may also be accomplished by chemical means, in which chemicals such as menthol, camphor, sorbitol and the like are applied to the affected tissues to transmit a sensation of cold to the area. Cooling applications to treat sore muscles, hemorrhoids and to relieve arthritic pain are known. However, such combinations may be extremely painful if inappropriately applied directly to sensitive, inflamed vaginal and vulvar tissue.

Currently, there are no known cooling preparations available or known to the art that are intended for vaginal, vulvar, introitus or labial use. Thus, one of the objects of this invention is to provide compositions and methods of treating vaginal infections that utilize cooling sensation to provide relief to itching, burning and pain in vaginal infections. Thus, in accordance with this invention, cooling compositions appropriate for the infected vaginal area may be used for treatment of burning, pain and discomfort associated with vaginal infections. Such compositions may be in the forms of creams, gels or the like semisolid preparations, that are capable of being applied topically and spread over the external portion of the vagina and the vulva. Such compositions may contain cooling ingredients including lower alcohols, menthol, camphor, sugars such as monosaccharides, disaccharides, oligosaccharides or polysaccharides or the like.

Such cooling ingredients may be combined in the cooling compositions of this invention with antimicrobial active ingredients including antifungals, antibacterials, antivirals and the like. Antifungal ingredients may include azoles, more preferably imidazoles and more specifically, miconazole nitrate, clotrimazole, econazole, ,albaconazole, ravuconazole, saperconazole, terconazole, ketoconazole, butaconazole, butaconazole, tioconazole, fluconazole, secnidazole, metronidazole, vericonazole, fenticonazole, sertaconazole, posaconazole, bifonazole, oxiconazole, sulconazole, elubiol, vorconazole, isoconazole, flutrimazole and their pharmaceutically acceptable salts and the like. Other antifungal agents may include an allylamine or one from other chemical families, including but not limited to, ternafine, naftifine, amorolfine, butenafine, ciclopirox, griseofulvin, undecyclenic acid, haloprogin, tolnaftate, nystatin, iodine, rilopirox, BAY 108888, purpuromycin and their pharmaceutically acceptable salts.

Another embodiment of the invention are compositions for vulvovaginal use containing one or more antibiotics. The antibiotic may be chosen from the group including, but not limited to, metronidazole, clindamycin, tinidazole, ornidazole, secnidazole, refaximin, trospectomycin, purpuromycin and their pharmaceutically acceptable salts and the like. Antiviral active ingredients include Acyclovir, emtricitabine, ribavirin, adefovir, dipivioxil, tenefovir, retrovir, epivir, indinavir, lamivudin, emetricitabine, sequnavir, hydroxyurea, fosamprenavir and the like.

Another embodiment of the compositions of this invention include compositions for vulvovaginal use containing one or more antiviral agents. Antiviral agents may preferably include, but are not limited to, immunomodulators, more preferably imiquimod, its derivatives, podofilox, podophyllin, interferon alpha, reticolos, cidofovir, nonoxynol-9 and their pharmaceutically acceptable salts and the like. Other active ingredients may include What about vaginal microbicides such as surfactants and other materials such as carrageenan, cellulose sulfate and sodium lauryl sulfate and the like.

External topical compositions of this invention may also contain skin protectants. By protecting the skin, not only does the composition soothe the site of infection; it also maintains the integrity of the skin to prevent additional damage and pain. Skin protectants may include allantoin, cocoa butter, dimethicone, kaolin, shark liver oil, petrolatum, vegetable oils, zinc oxide and others known to those of skill in the art.

Local anesthetics or antihistamines may also be employed in the external topical compositions of this invention in order to lessen the pain and itching caused by the local infection. Local anesthetics and antihistamines that are useful in the compositions of this invention include benzocaine, lidocaine, dibucaine, benzyl alcohol, camphor, resorcinol, menthol and diphenhydramine hydrochloride and the like.

Anti-inflammatories such as corticosteroids, including hydrocortisone acetate, may also be employed in the external topical compositions of this invention. COX 2 Inhibitors may also be used, such as Valdecoxib, Celocoxib and Refecoxib. Non-steroidal anti-inflammatory drugs (NSAIDS) such as Indomethacin, Naproxen Sodium, Naproxen Potassium, Diclofenac sodium, Oxaproxin, Salicylate, Etodolac, Meloxicam, Ketoprofen, Tolmecytin sodium, Choline Magnesium and Trisalicylate may also be useful in the compositions and devices of this invention.

The external topical compositions of this invention may be in the form of emulsions such as creams, lotions, ointments, powders, microemulsions, liposomes or may be gels and liquids. Emulsions may include oil in water or water in oil emulsions. The external topical compositions of this invention may also include intravaginal dosage forms such as creams, ointments, gels, gelatin capsules, suppositories and the like.

The cooling compositions of this invention may be applied manually, directly by the user with her fingers or hand or using a glove or cot. The cooling compositions of this invention may be applied with the application devices described in U.S. Pat. No. 6,156,323. Alternatively, the cooling compositions of this invention may be applied using a non-woven or woven fabric substrate. In accordance with this embodiment of the compositions of this invention, the substrate is impregnated with a liquid, cream, lotion or gel. The patient may use this impregnated substrate to wipe the external vaginal area, thereby removing vaginal discharge and simultaneously applying the cooling compositions of this invention.

In accordance with the methods of this invention, the patient should first clean the vaginal/vulvar area by applying a wipe to remove vaginal discharge. This wipe may contain the cooling composition of this invention. She should then administer her anti-infective medication, either by insertion into the vagina or orally (although if she is treating the infection systemically, she may, alternatively, administer the systemic medication prior to local treatment or cleansing). After cleaning the area, the patient should apply the cooling compositions of this invention in order to provide relief to the area and may simultaneously apply any local topical treatment that can offer anti-infective and/or analgesic properties to the area.

Another method may additionally have the step of applying a semisolid composition containing an active antifungal ingredient to the perineum after cleansing and inserting a dosage form containing an antifungal active ingredient into the vagina. The semisolid composition may preferably be in the form of a cream. More preferably, the antifungal active ingredient utilized in the semisolid composition as well as the dosage form is miconazole nitrate. Most preferably, the dosage form containing miconazole nitrate is contained in a soft gelatin dosage form.

According to the methods of this invention, a person may utilize the cooling compositions of this invention and regimen according to this invention to treat additional vulvovaginal conditions that cause pain and discomfort, such as vulvar vestibulitis, interstitial cystitis, vulvar vaginitis or vulvar dynea (vulvodynia). Compositions according to this invention useful in treating such conditions may contain cooling ingredients as well as anti-inflammatory active ingredients, ingredients that treat pain such as local anesthetics and other actives known to address such conditions, such as cromolyn sodium and the like.

Compositions that may be useful in accordance with this invention may contain from about 5 to about 20% w/w of a lower alkyl alcohol. More preferably, they should contain from about 5 to about 10% w/w of a lower alkyl alcohol. They may contain from about 5 to about 35% w/w of a polyol. Preferably, the polyol is a polyhydric alcohol, and more preferably, at least two polyhydric alcohols. Polyethylene glycol (hereinafter, “PEG”) ethers may also be used, including PEG ethers of propylene glycol, propylene glycol stearate, propylene glycol oleate and propylene glycol cocoate and the like. Specific examples of such PEG ethers include PEG-25 propylene glycol stearate, PEG-55 propylene glycol oleate and the like. Preferably, at least one of the polyhydric alcohols of the compositions of this invention is a polyalkylene glycols or others selected from the following group: glycerine, propylene glycol, butylene glycol, hexalene glycol or polyethylene glycol of various molecular weight and the like and/or combination thereof. More preferably, the compositions of this invention contain a polyethylene glycol; most preferably, the polyethylene glycol may be selected from the following group: polyethylene glycol 400 or polyethylene glycol 300. Polypropylene glycol of various molecular weights may also be used. PEGylated compounds such as peptide or protein derivatives obtained through PEGylation reactions may also be used. In addition, block copolymers of PEG's may be used, such as (ethylene glycol)-block-poly(propylene glycol)-block-(polyethylene glycol), poly(ethylene glycol-ran-propylene glycol) and the like. The compositions of this invention should contain polyhydric alcohols in an amount from about 80% to about 98% by weight of the composition.

Preferred embodiments include those in which the compositions of this invention are in the form of a cooling gel such as a hydroalcohlic gel includes water and ethyl alcohol and additional soothing components. This cooling gel composition may also contain a local anesthetic such as benzocaine, lydocaine, dibucaine, tetracaine, diphenyhydramine hydrochloride, tripelennamine, hydrochloride, praxomoxine hydrochloride, butamben picrate and resorcinol. Cooling gel compositions of this invention may further contain anti-inflammatory compounds such as hydrocortisone and others known to the art. Menthol, sorbitol, camphor or other chemical agents may also be included in order to convey to the patient a sensation of cooling.

Compositions of this invention may also include plant extracts such as aloe, witch hazel, chamomile, hydrogenated soy oil and colloidal oatmeal, and vitamins such as vitamins A, D or E or the like.

The compositions of this invention may also be made in the form of an oil-in-water cream or lotion. Such oil-in-water creams or lotions may contain menthol, sorbitol, camphor or other chemical agents to convey a sensation of cooling. They may also contain a local anesthetic such as benzocaine, lidocaine, dibucaine, tetracaine, diphenyhydramine hydrochloride, tripelennamine, hydrochloride, praxomoxine hydrochloride, butamben picrate and resorcinol. The oil-in-water compositions of this invention may further include anti-inflammatory compounds such as hydrocortisone and others known to those of ordinary skill in the art.

Compositions of this invention may alternatively be made in the form of a hydroalcoholic liquid that contains water and a lower alkyl alcohol such as, preferably, ethyl alcohol, and additional soothing components. Such compositions can be used to coat or impregnate a suitable fabric to be used as a vaginal wipe.

The following examples serve to illustrate, but not limit, the scope of the inventions described herein.

### EXAMPLE 1

Composition 1. (Liquid)

| | |
|---|---|
| 70% Ethyl alcohol (SDA 40) | 5.00% |
| Propylene Glycol | 5.00% |
| Sorbitol Solution | 5.00% |
| Purified water | 85.00% |

This composition may be used to impregnate a fabric wipe.

Composition 2. (Gel)

| | |
|---|---|
| 70% Ethyl alcohol (SDA 40) | 5.00% |
| Propylene Glycol | 5.00% |
| Sorbitol Solution | 5.00% |
| Hydroxyethylcellulose | 1.50% |
| Purified water | 83.50% |

This composition may be used to impregnate a fabric wipe.

Composition 3. (Gel)

| | |
|---|---|
| 70% Ethyl alcohol (SDA 40) | 5.00% |
| Propylene Glycol | 5.00% |
| Sorbitol Solution | 5.00% |
| Hydroxyethylcellulose | 1.50% |
| Benzocaine | 2.00% |
| Purified water | 81.50% |

This composition may be used to impregnate a fabric wipe.

Composition 4. (Gel)

| | |
|---|---|
| 70% Ethyl alcohol (SDA 40) | 5.00% |
| Propylene Glycol | 5.00% |
| Sorbitol Solution | 5.00% |
| Hydroxyethylcellulose | 1.50% |
| Hydrocortisone | 2.00% |
| Purified water | 81.50% |

This composition may be used to impregnate a fabric wipe.

Composition 5. (Liquid)

| | |
|---|---|
| Menthol | 1.00% |
| Propylene Glycol | 5.00% |
| Sorbitol Solution | 5.00% |
| Purified water | 89.00% |

This composition may be used to impregnate a fabric wipe.

Composition 6. (Gel)

| | |
|---|---|
| Menthol | 1.00% |
| Propylene Glycol | 5.00% |
| Sorbitol Solution | 5.00% |
| Hydroxyethylcellulose | 1.50% |
| Purified water | 87.50% |

This composition may be used to impregnate a fabric wipe.

Composition 7. (Gel)

| | |
|---|---|
| Menthol | 1.00% |
| Propylene Glycol | 5.00% |
| Sorbitol Solution | 5.00% |
| Hydroxyethylcellulose | 1.50% |
| Benzocaine | 2.00% |
| Purified water | 85.50% |

This composition may be used to impregnate a fabric wipe.

Composition 8. (Gel)

| | |
|---|---|
| Menthol | 1.00% |
| Propylene Glycol | 5.00% |
| Sorbitol Solution | 5.00% |
| Hydroxyethylcellulose | 1.50% |
| Hydrocortisone | 2.00% |
| Purified water | 85.50% |

This composition may be used to impregnate a fabric wipe.

Composition 9. (Cream)

| | |
|---|---|
| Miconazole Nitrate | 2.00% |
| Propylene Glycol | 20.00% |
| Cetyl Alcohol | 3.00% |
| Stearyl Alcohol | 8.50% |
| Isopropyl Myristate | 1.00% |
| Polysorbate 60 | 3.00% |
| Benzoic Acid | 0.20% |
| Lactic Acid | 0.02% |
| Menthol | 1.00% |
| Purified water | 61.28% |

This composition may be used to impregnate a fabric wipe.

Composition 10. (Lotion)

| | |
|---|---|
| Miconazole Nitrate | 2.00% |
| Propylene Glycol | 20.00% |
| Cetyl Alcohol | 1.00% |
| Stearyl Alcohol | 5.50% |
| Isopropyl Myristate | 1.00% |
| Polysorbate 60 | 3.00% |
| Benzoic Acid | 0.20% |
| Lactic Acid | 0.02% |
| Menthol | 1.00% |
| Purified water | 66.28% |

This composition may be used to impregnate a fabric wipe.

Composition 11. (Cream)

| | |
|---|---|
| Propylene Glycol | 20.00% |
| Cetyl Alcohol | 3.00% |
| Stearyl Alcohol | 8.50% |
| Isopropyl Myristate | 1.00% |
| Polysorbate 60 | 3.00% |
| Benzole Acid | 0.20% |
| Lactic Acid | 0.02% |
| Menthol | 1.00% |
| Purified water | 63.28% |

This composition may be used to impregnate a fabric wipe.

Composition 12. (Lotion)

| | |
|---|---|
| Propylene Glycol | 20.00% |
| Cetyl Alcohol | 1.00% |
| Stearyl Alcohol | 5.50% |
| Isopropyl Myristate | 1.00% |
| Polysorbate 60 | 3.00% |
| Benzoic Acid | 0.20% |
| Lactic Acid | 0.02% |
| Menthol | 1.00% |
| Purified water | 68.28% |

This composition may be used to impregnate a fabric wipe.

1. A method of treating a vaginal infection comprising cleaning the perineum of a woman, cooling the perineum and applying to the infected area an active compound to treat the infection.

2. A method according to claim 1 wherein said cooling step comprises applying a wet substrate to the infected area.

3. A method according to claim 1 wherein said cooling step comprises applying to the affected area a composition comprising from about 5 to about 20% w/w of a lower alkyl alcohol.

4. A method according to claim 3 wherein said cooling step comprises applying to the affected area a composition comprising from about 5 to about 10% w/w of a lower alkyl alcohol.

5. A method according to claim 4 wherein said cooling step comprises applying to the affected area a composition comprising They may contain from about 5 to about 35% w/w of a polyol.

6. A method according to claim 3 wherein said composition further comprises an antifungal compound.

7. A method according to claim 6 wherein said antifungal compound is selected from the group consisting of: miconazole nitrate, clotrimazole, econazole, ,albaconazole, ravuconazole, saperconazole, terconazole, ketoconazole, butaconazole, butaconazole, tioconazole, fluconazole, secnidazole, metronidazole, vericonazole, fenticonazole, sertaconazole, posaconazole, bifonazole, oxiconazole, sulconazole, elubiol, vorconazole, isoconazole, flutrimazole and their pharmaceutically acceptable salts, ternafine, naftifine, amorolfine, butenafine, ciclopirox, griseofulvin, undecyclenic acid, haloprogin, tolnaftate, nystatin, iodine, rilopirox, BAY 108888, purpuromycin and their pharmaceutically acceptable salts.

8. A method according to claim 3 wherein said composition further comprises an antibiotic.

9. A method according to claim 8 wherein said antibiotic is selected from the group consisting of: metronidazole, clindamycin, tinidazole, ornidazole, secnidazole, refaximin, trospectomycin, purpuromycin and their pharmaceutically acceptable salts.

10. A method according to claim 3 wherein said composition further comprises an antiviral compound.

11. A method according to claim 10 wherein said antiviral compound Acyclovir, emtricitabine, ribavirin, adefovir, dipivioxil, tenefovir, retrovir, epivir, indinavir, lamivudin, emetricitabine, sequnavir, hydroxyurea and fosamprenavir.

12. A method according to claim 3 wherein said composition comprises lower alcohols, menthol, camphor and sugars.

13. A method according to claim 12 wherein said sugar is selected from the group consisting of monosaccharides, disaccharides, oligosaccharides or polysaccharides.

14. A method according to claim 12 wherein said sugar is sorbitol.

15. A method according to claim 1 wherein said cleaning and cooling steps comprise applying a wet substrate to the perineum and said treating step comprises inserting an antifungal active ingredient into the vagina.

16. A method according to claim 15 wherein said method further comprises the step of applying a semisolid composition comprising an active antifungal ingredient to the perineum.

17. A method according to claim 16 wherein said semisolid composition is a cream.

18. A method according to claim 15 wherein said antifungal active ingredient is miconazole nitrate.

19. A method according to claim 16 wherein said antifungal active ingredient is miconazole nitrate.

20. A method according to claim 19 wherein said miconazole nitrate is contained in a soft gelatin dosage form.

21. A method of treating vulvar vestibulitis, interstitial cystitis, vulvar vaginitis or vulvar dynea comprising applying to the perineum of a woman a composition comprising a cooling agent.
